(19) United States
(12) Patent Application Publication
(10) Pub. No.: US 2008/0249602 A1
(43) Pub. Date: Oct. 9, 2008

(54) Artificial Blood Vessel Stent

(76) Inventors: Sung Gwon Kang, Gyeonggi-do (KR); Eun Sang Kim, Seoul (KR)

Correspondence Address: BIRCH STEWART KOLASCH & BIRCH, PO BOX 747, FALLS CHURCH, VA, 22040-0747, US

(21) Appl. No.: 11/579,338

(22) PCT Filed: Oct. 12, 2004
(86) PCT No.: PCT/KR2004/002594

§ 371 Date: Nov. 1, 2006

(30) Foreign Application Priority Data
Jun. 15, 2004 (KR) 10-2004-0043825

(51) Int. Cl. A61F 2/06

(52) U.S. Cl. 623/1.15; 623/1.2; 623/1.36

(57) ABSTRACT

The present invention discloses an artificial blood vessel stent, which can minimize the diameter of a stent insertion device because the diameter (profile) thereof is uniform and small over the entire length upon being loaded, and accordingly can be easily placed in a branched portion of a blood vessel using a puncture method. The artificial blood vessel stent comprises: (I) an artificial blood vessel body 1 in which the upper end part is formed with a single cylindrical shape or partially cylindrical shape and the lower end part is divided into two small cylindrical shapes or partially cylindrical shapes having different lengths; (II) a cylindrical first stent member 2 connected to the upper end of the artificial blood vessel body and being expandable; (III) a cylindrical second stent member 3 connected to the shorter side of the lower end part of the artificial blood vessel body and being expandable; and (IV) a cylindrical third stent member 4 inserted into the longer side of the lower end part of the artificial blood vessel body, so that the upper end surface coincides with the lower end surface of the second stent member, and being expandable.

### TECHNICAL FIELD

The present invention relates to an artificial blood vessel stent, and more particularly, to an artificial blood vessel stent which can minimize the diameter of a stent insertion device (hereinafter, ‘cannula device’) because the diameter (profile) of the stent is uniform and small over the entire length thereof upon being contracted and inserted (loaded) into the insertion device, and accordingly can be easily placed in a branched portion of a blood vessel, such as an artery, using a puncture method.

As an example, aneurysm is characterized by swelling of an artery, similar to the way in which a balloon expands, and is a very serious disease since weakened arterial walls may rupture if intravascular pressure increases above a certain level.

### BACKGROUND ART

As one method of treating aneurysm, an artificial blood vessel stent has been developed which can solve the problems associated with aneurysm through a surgical operation and minimize patient's pains without an operation when a surgical operation is not applicable to the patient. However, due to the fact that the size of a cannula device used for such a stent must be expanded to a diameter of more than 6 mm, a cut-down method, in which a blood vessel is locally surgically opened followed by insertion of the stent, has been mainly employed.

Although the said cut-down method is more convenient than full surgical operation, it is still inconvenient in that a local operation must be performed. Furthermore, it is very difficult to insert the stent if the blood vessel is curved or twisted. Thus, the puncture method was developed and popularized.

The said puncture method is a treatment method in which a fine hole is made in a blood vessel, particularly, in an artery, without operation locally opening blood vessel, a stent cannula device then being inserted into the artery through the hole, and an artificial blood vessel stent then placed at the lesion region of the artery by means of the stent cannula device inserted into the fine hole.

In order to use the puncture method, the inner diameter of the stent cannula device needs to be lower than, for example, 4 mm, and the outer diameter thereof needs to be lower than, for example, 4.5 mm.

Moreover, the artificial blood vessel stent has to be contracted to a size smaller than, for example, the inner diameter of the cannula device so that it can be inserted into the stent cannula device.

As shown in FIG. 1, however, a conventional artificial blood vessel stent has a monolithic structure in which an inner bare stent is inserted into an artificial blood vessel body and formed integrally therewith. Thus, the diameter (profile) of the artificial blood vessel stent contracted upon being inserted (loaded) into the stent cannula device is more than 4.5 mm.

Due to this, the outer diameter of the stent cannula device used to place the conventional artificial blood vessel stent as shown in FIG. 1 must be larger than 4.95 mm. Thus, it is impossible to place the conventional artificial blood vessel stent in a blood vessel by the puncture method.

In FIG. 1, illustration of the middle portion of the inner bare stent inserted into the artificial blood vessel body is omitted.

### DISCLOSURE OF THE INVENTION

The present invention has been proposed to solve the above problems, and provides an artificial blood vessel stent which can minimize the diameter of a stent cannula device to less than 4.5 mm because the diameter (profile) of the stent is uniform and small over the entire length thereof upon separation from an inner bare stent and being loaded into the cannula device, and accordingly can be placed in blood vessels using a puncture method.

Furthermore, the present invention provides an artificial blood vessel stent which is suitable for placement into branch points in blood vessels, such as arteries, because it has an inverted Y-shaped structure.

### BEST MODE FOR CARRYING OUT THE INVENTION

Hereinafter, a preferred embodiment of the present invention will be described in detail with reference to the accompanying drawings.

As shown in FIG. 2, an artificial blood vessel stent of the present invention comprises:

an artificial blood vessel body 1 which is sufficiently flexible to be bent in any direction and has a structure in which the upper end part thereof is formed with a single cylindrical shape or partially cylindrical shape and the lower end part thereof is divided into two small cylindrical shapes or partially cylindrical shapes having different lengths, thereby being capable of insertion into a blood vessel and preventing the intravascular pressure from being applied to weakened walls of the blood vessel;

a cylindrical first stent member 2 that is connected to the upper end of the artificial blood vessel body to support the artificial blood vessel body, and is contractible to a certain size and has a certain degree of expandability;

a cylindrical second stent member 3 that is connected to the shorter side of the lower end part of the artificial blood vessel body to support the shorter side of the lower end part of the artificial blood vessel body, and is contractible to a certain size and has a certain degree of expandability; and

a cylindrical third stent member 4 that is inserted into the longer side of the lower end part of the artificial blood vessel body so that the upper end surface coincides with the lower end surface of the second stent member to thus support the longer side of the lower end part of the artificial blood vessel body, and is contractible to a certain size and has a certain degree of expandability.

Further, a plurality of barbs 5 for fixing the artificial blood vessel stent to a blood vessel are placed at the region connecting the artificial blood vessel body 1 and the first stent member 2.

The barbs are metal wires, whose middle parts are twisted and fixed to the first stent member 2, and having both opposite end parts thereof protruded to the outside. They play the role of preventing the artificial blood vessel stent from displacement (migration) due to the force of blood pressure applied thereto after initial placement of the artificial blood vessel stent.

The first stent member 2, second stent member 3 and third stent member 4 may have identical or different structures, and it is preferred that each of them forms at least one turn so as to have a cylindrical shape.

The artificial blood vessel body 1 is made of a high density fabric which is sufficiently flexible to be bent in any direction, and the first stent member 2 and the third stent member 4 are made of metal wire or the like being contractible to a predetermined size and having a certain degree of expandability.

As shown in FIG. 1. the artificial blood vessel stent of the present invention has a structure in which the lower end of the artificial blood vessel body 1 is divided into two cylindrical shapes or partially cylindrical shapes having a different length so that the diameter (profile) becomes uniform over the entire length upon being inserted (loaded) into the stent cannula device. The second stent member 3 is connected (not inserted) to the shorter side of the lower end part of the artificial blood vessel body 1, and the third stent member 4 is inserted into the longer side of the lower end part of the artificial blood vessel body 1 so that the upper end surface coincides with the lower end surface of the second stent member.

Further, the artificial blood vessel stent of the present invention, rather than being formed integrally with the inner bare stent, is separated therefrom so as to allow the diameter thereof to be reduced upon being inserted (loaded) into the stent cannula device.

Due to this, the artificial blood vessel stent of the present invention is small and uniform over the entire length, i.e., the diameter thereof is less than 4.0 mm upon being inserted (loaded) into the stent cannula device.

As a result, the artificial blood vessel stent can be placed in a blood vessel using the puncture method since the inner diameter and outer diameter of the stent cannula device can be minimized.

In particular, in the present invention, the inner diameter of the stent cannula device can become less than 4.0 mm and the outer diameter thereof can become less than 4.5 mm.

As shown in FIGS. 5 and 6, the artificial blood vessel stent of the present invention can be inserted and placed into a blood vessel by a typical stent cannula device comprising a cannula device member 10 for receiving the artificial blood vessel stent, a pusher member 20 for pushing the artificial blood vessel stent out of the cannula device member 10 and a catheter 30 having a streamlined tip part 31 formed on the front end thereof and being placed in the pusher member.

The procedure for placing the artificial blood vessel stent in accordance with the present invention inside a blood vessel by using the stent cannula device will be described below.

Firstly, as shown in FIG. 5, the artificial blood vessel stent of the present invention is contracted and inserted into the front end of the cannulation member 10 of the stent cannula device, and then the front end of the cannulation member of the stent cannula device is inserted at the location of a vascular lesion portion of a patient by using a guide member (not shown) inserted into the catheter 30.

In this state, if the cannulation member 10 is pulled backward with the pusher member 20 fixed, as shown in FIG. 6, the artificial blood vessel stent expands on account of its own elasticity upon being pushed out of the cannulation member 10, and is fixedly positioned in the blood vessel.

FIG. 5 is a cross sectional view of the artificial blood vessel stent of the present invention inserted into the cannula device. FIG. 6 is a cross sectional view of the artificial blood vessel stent of the present invention in the process of being pushed out of the cannula device.

A concrete description of how the artificial blood vessel stent of the present invention may be used to treat an aneurysm occurring at a branch-portion of a blood vessel will be given below.

As seen from the above, the artificial blood vessel stent of the present invention is placed in a blood vessel using the stent cannula device, and then the inner bare stent as shown in FIG. 3 is inserted into the upper end part of the artificial blood vessel stent body 1 placed in advance in the blood vessel by the same method by using another stent cannula device. Subsequently, the two limb stents as shown in FIG. 4 are inserted into the second stent 3 and third stent 4 of the artificial blood vessel stent placed in advance in the blood vessel by the same method by using other stent cannula devices respectively, thereby completing the treatment of aneurysm of the blood vessel-branched portion.

FIG. 4 is a front view of a limb stent whose middle part is cut out, wherein illustration of the inner bare stent inserted into the middle part is omitted.

### ADVANTAGEOUS EFFECT

The artificial blood vessel stent of the present invention can minimize the diameter of a stent cannula device because the diameter of the stent is small and uniform over the entire length thereof upon being separated from an inner bare stent and loaded into the cannula device, and accordingly is able to be easily placed in a blood vessel using a puncture method.

Moreover, the artificial blood vessel stent of the present invention is more suitable for placement into a branched portion of a blood vessel, such as an artery, due to the inverted Y-shaped structure thereof.

### BRIEF DESCRIPTION OF THE DRAWINGS

FIG. 1 is a front view of a conventional artificial blood vessel stent;

FIG. 2 is a front view of an artificial blood vessel stent in accordance with the present invention;

FIG. 3 is a front view of an inner bare stent which is inserted into a top part of an artificial blood vessel body 1 of the artificial blood vessel stent of the present invention by another cannular device;

FIG. 4 is a front view of a limb stent that is inserted into a lower part of an artificial blood vessel 1 of the artificial blood vessel stent of the present invention by another cannular device;

FIG. 5 is a cross sectional view of the artificial blood vessel stent of the present invention inserted into the cannular device; and

FIG. 6 is a cross sectional view showing the artificial blood vessel stent of the present invention in the process of being pushed out of the cannular device.

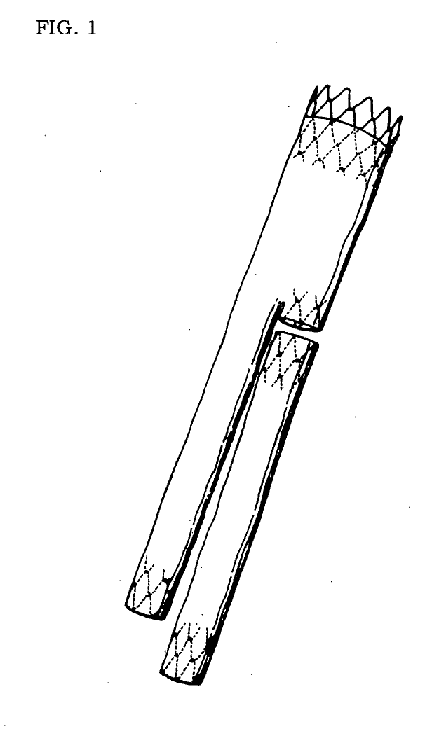

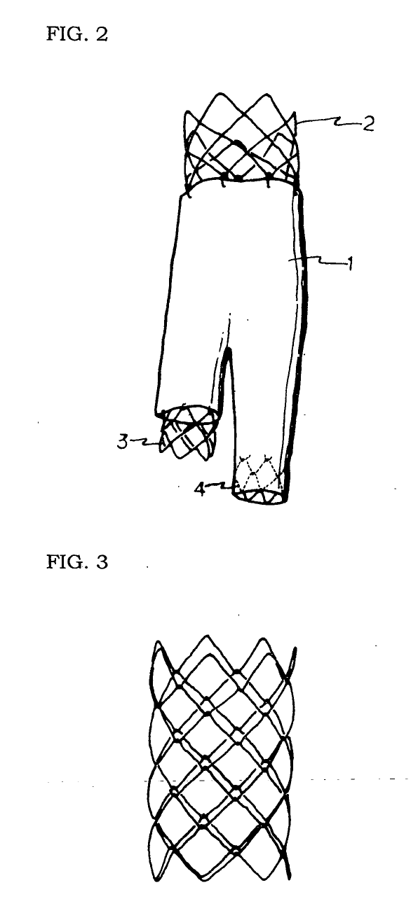

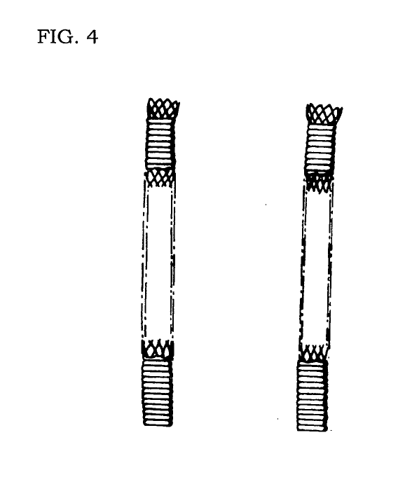

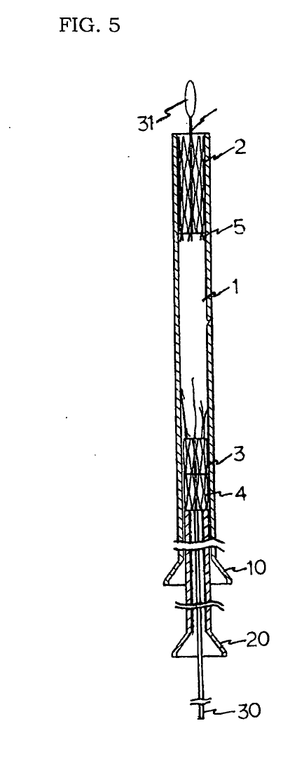

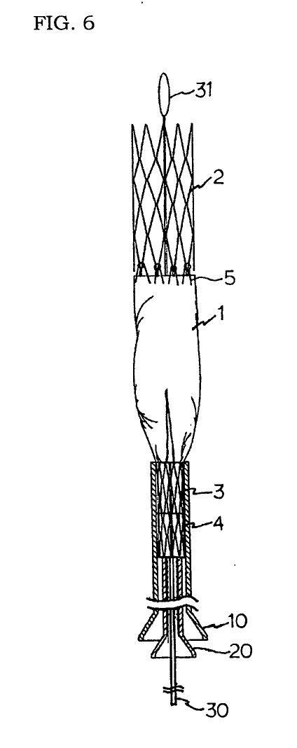

### INDUSTRIAL APPLICABILITY

The artificial blood vessel stent of the present invention is applicable to the treatment of arterial diseases since it can be easily inserted and placed in a branched portion of a blood vessel.

What is claimed is:

1. An artificial blood vessel stent, comprising:
an artificial blood vessel body 1 which is sufficiently flexible to be bent in any direction and has a structure in which the upper end part thereof is formed with a single cylindrical shape or partially cylindrical shape and the lower end part thereof is divided into two small cylindrical shapes or partially cylindrical shapes having different lengths, thereby being capable of insertion into a blood vessel and preventing the intravascular pressure from being applied to weakened walls of the blood vessel;
a cylindrical first stent member 2 that is connected to the upper end of the artificial blood vessel body to support the artificial blood vessel body, and is contractible to a certain size and has a certain degree of expandability;
a cylindrical second stent member 3 that is connected to the shorter side of the lower end part of the artificial blood vessel body to support the shorter side of the lower end part of the artificial blood vessel body, and is contractible to a certain size and has a certain degree of expandability; and
a cylindrical third stent member 4 that is inserted into the longer side of the lower end part of the artificial blood vessel body so that the upper end surface coincides with the lower end surface of the second stent member to thus support the longer side of the lower end part of the artificial blood vessel body, and is contractible to a certain size and has a certain degree of expandability.

2. The artificial blood vessel stent of claim 1, wherein a plurality of barbs 5 for fixing the artificial blood vessel stent to a blood vessel are placed at the region connecting the artificial blood vessel body 1 and the first stent member 2.

3. The artificial blood vessel stent of claim 1, wherein the artificial blood vessel stent is uniform over the entire length thereof, and has less than 4.0 mm of the diameter upon being inserted (loaded) into the stent cannula device.

4. The artificial blood vessel stent of claim 1, wherein the first stent member 2, second stent member 3, and third stent member 4 have identical or different structures.

5. The artificial blood vessel stent of claim 1, wherein the first stent member 2, second stent member 3 and third stent member 4 form at least one turn so as to have a cylindrical or partially-cylindrical shape.

6. The artificial blood vessel stent of claim 1, wherein the artificial blood vessel body 1 is made of a high density fabric.

7. The artificial blood vessel stent of claim 1, wherein the first stent member 2, second stent member 3 and third stent member 4 are made of metal wire.
